(19) United States
(12) Patent Application Publication
(10) Pub. No.: US 2010/0189871 A1
(43) Pub. Date: Jul. 29, 2010

(54) BIOTOXIN SORBENT AND METHOD FOR PREPARING THE SAME

(75) Inventors: Xuefeng Yu, Hubei (CN); Zhihong Li, Hubei (CN); Minghua Yu, Hubei (CN); Juan Yao, Hubei (CN); Bin Tan, Hubei (CN); Jinlin Zhu, Hubei (CN)

Correspondence Address: CLARK & ELBING LLP, 101 FEDERAL STREET, BOSTON, MA, 02110, US

(73) Assignee: Angel Yeast Co., Ltd, Yichang (CN)

(21) Appl. No.: 12/668,564

(22) PCT Filed: Aug. 6, 2008
(86) PCT No.: PCT/CN2008/001433

§ 371 Date: Jan. 27, 2010

(30) Foreign Application Priority Data
Aug. 10, 2007 (CN) 200710135740.6

(51) Int. Cl. A23K 1/175; C12P 19/04; B01J 20/12; B01J 20/22

(52) U.S. Cl. 426/623; 435/101; 426/630; 502/404

(57) ABSTRACT

The present invention provides a combination used for absorbing toxins in feed, comprising 1-10% by weight of clay and 90-99% by weight of yeast manna oligosaccharide, wherein the yeast manna oligosaccharide is extract of yeast cell wall which is separated from Saccharomyces cerevisiae. The biotoxin sorbent product can be used to feed any animals, including livestock, fowls, marine lives and ruminants. When mixing with feed, owing to its strong toxin adsorption capability, this absorbent may reduce the amount of toxins entering bodies of animals, thereby improving productivity and health of animals, reducing diseases caused by toxins.

### THE FIELD OF THE INVENTION

The present invention relates to a biotoxin sorbent and a preparing method thereof.

### BACKGROUND OF THE INVENTION

Every year, a large amount of foodstuff used for animal feeds is contaminated by toxins generated in mildew (mycotoxins). Mildews, such as aspergillus niger, aspergillus flavus and blue mold, generated during storage of foodstuff or other feed raw materials often result in decreased nutritive valve of feed and alimentary toxicosis of animals.

Mycotoxinmay may consequently affect adversely productive ability and health of animals.

Mycotoxin has many harmful effects, such as reducing feed intake, decreasing feed conversion rate and slowing down animal's growth rate. Mycotoxin also does harm to human's healthy because mycotoxin is introduced in meat and milk due to animal's diet intake.

The acute symptoms caused by mycotoxin are relatively easy to determine. Whereas, chronic symptoms such as slight reduction of productivity and/or immunosuppression are usually inconspicuous but may induce big economic loss. Therefore, people, especially in the livestock industry, have to find out viable treatment methods to use feed that has been polluted by mildew due to economic reason.

There are many methods for removing mycotoxin from feed, including blend of contaminated and uncontaminated feeds; physical separation for removing heavily polluted feed and ammoniation or heating for detoxification. However, these traditional methods have disadvantages of high labor intensity, low economical efficiency and invalidity to certain kinds of mildew.

A more viable method is adding into feed a material capable of absorbing toxin so as to prevent toxins from entering blood vessels of animals. Many chemicals have been proved to be useful in commercial applications, in which mineralogical clay is well known as an sorbent.

U.S. Pat. No. 5,149,549 disclosed a use of kaolinite, a particular type of bentonite, which is mixed with feed as a toxin sorbent.

Chinese patent CNO2111097.2 disclosed a method for preparing a nanometer feed additive that can absorb mycotoxin in a feed. This method includes the following steps: adding water to a mixture of nanometer montmorillonite and sodium chloride by a determined ratio and stirring to form a slurry; then adding glucomannan into the slurry to produce a toxin sorbent.

However, there are certain limits to using clay as a toxin sorbent. Firstly, such a toxin sorbent must be used at a high concentration in order to obtain obvious absorption effect. Secondly, the sorbent has a limited scope of usage because most kinds of sorbents have substantial absorption effect only on flavacin toxin.

Recently, glucomannan has been proposed as a toxin sorbent. Although glucomannan has some effect of biological absorption, most of glucomannan is extracted from plants such as Amorphophallus konjac, resulting in poor solubility. The limited solubility (in water) leads to its insufficient contact with mycotoxin and bad absorption effect to wide-spectrum moulds (mildew).

Accordingly, a novel toxin sorbent with good miscibility with feeds and high effectiveness in absorbing various toxins is needed,

### SUMMARY OF THE INVENTION

The present invention generally provides a method for combining and deactivating mycotoxin in animal feeds.

A further objective of the invention is to provide a method for combining extract of yeast cell wall with mineralogical clay (for example, zeolite powder, bentonite, aluminosilicate), to absorb and deactivate mycotoxin in animal feeds.

Another objective of the invention is to provide a toxin sorbent that can mix with animal feeds and functions well at a lower concentration than prior products.

To achieve above objectives, this invention provides a biotoxin sorbent comprising 1-10%, preferably 4-8%, more preferably 5-7% by weight of clay and 90-99%, preferably 92-96%, more preferably 93-95% by weight of yeast manna oligosaccharide, wherein the yeast manna oligosaccharide is prepared from Saccharomyces cerevisiae strain.

The present invention further provides a method for preparing above biotoxin sorbent, comprising steps of:
providing yeast cell wall;
preparing manna oligosaccharide from the yeast cell wall by:
i) boiling the yeast cell wall in a boiling vessel for 1 to 2 hours at 80-98° C.;
ii) enzymolyzing the yeast cell wall by using a biological enzyme selected from alkaline proteinase, mannanase and amy lase;
iii) centrifuging to obtain enzymatic hydrolysates;
iv) concentrating the resultant clear liquid to obtain a concentrate; and
v) drying the concentrate to produce a manna oligosaccharide dried powder; and

mixing the manna oligosaccharide dried powder with the clay uniformly.

In the method, the yeast cell wall may be obtained from commercially marketed Saccharomyces cerevisiae, or from Saccharomyces Cerevisiae Strain Z2.2 through scale-up culturing a Saccharomyces cerevisiae strain using fermentable sucrose molasses and dissolving the resultant yeast cells, wherein Strain Z2.2 (Angle's deposit number), belonging to Saccharomyces cerevisiae Hanse, is sieved by the applicant with M205128 as its deposit number of China Center for Type CultureCollection.

The biotoxin sorbent according to the invention can be used to feed any animals, including livestocks, fowls, marine lives, cud chewers and so on. When mixing with feed, owing to its strong toxin adsorption capability, the sorbent may reduce the amount of toxins entering bodies of animals, thereby improving productivity and health of animals and reducing diseases caused by toxins. The biotoxin sorbent prepared in the invention can also be used in storage of daily diet.

### DETAIL OF THE INVENTION

### DEFINITION

“Combination” means herein a product which is prepared by combining extract of yeast cell wall, i.e. manna oligosaccharide, with clay, the combination is a toxin sorbent which is provided according to objectives of the invention, so sometimes it is called toxin sorbent.

The inventor surprisingly discovered that the extract of yeast cell wall has excellent function of absorbing toxins in feed after combining with metallogenic clay. Thus, the present invention provides a combination of yeast manna oligosaccharide and metallogenic clay and a method for preparing such a combination.

The clay used in the invention can be any kind of products that are commercially available and can be added into feed. The clay includes, but not limited to, zeolite, bentonite and aluminosilicate. A typical kind of clay is aluminosilicate with particle diameter size in the range of 10-100 microns.

The extract of yeast cell walls used in the invention may be prepared from any yeast cell including Brewer's yeast, Saccharomyces cerivisiae and baker's yeast.

In a typical embodiment of the invention, the yeast manna oligosaccharide is prepared from Saccharomyces cerevisiae strain Z2.2 (Angel's deposit number), which is specially cultivated by the applicant, and with its species name of Latin being Saccharomyces cerevisiae Hanse. The strain has been deposited in China Center for Type Culture Collection (CCTCC) (Wuhan University) since Oct. 25, 2005 with the deposit number of M205128. The yeast manna oligosaccharide is in the form of a gray tasteless powder and hydrosoluble. The preparation thereof will be described in detail later.

The combination according to the invention preferably includes 1-10% by weight of clay and 90-99% by weight of yeast manna oligosaccharide, more preferably 4-8% by weight of clay and 92-96% by weight of yeast manna oligosaccharide, most preferably 5-7% by weight of clay and 93-95% by weight of yeast manna oligosaccharide.

To obtain the combination of the invention, the yeast manna oligosaccharide may firstly be mixed with clay by traditional means so as to maintain uniformity of the components in the combination.

The combination prepared by the above method is preferably in the form of a dry free-flowing powder so that the combination is easy to add to feed directly or mix with diet as additive.

The preparation of the extract of yeast cell walls, yeast manna oligosaccharide will be described in detailed hereinafter.

### Production of Yeast Cell Walls

The extract of yeast cell wall used in the invention may be prepared from marketed yeast cell. It may also be prepared from Strain Z2.2 cultivated by the applicant. In the preparation, several scale-up culturing are carried out to enrich active yeast, during which treated fermentable sucrose molasses are used as liquid medium. Then, the obtained mixture is centrifuged and washed, to forming a yeast cream. Strain Z2.2cultivated by the applicant has been deposited at China Center for Type Culture Collection (CCTCC) (Wuhan University) since Oct. 25, 2005, and the deposit number is given as M205128. The scale-up culturing is carried out in a culture solution of 12 Brix, at 30° C., pH 4.2-5.4 with no ventilation made in the initial stage and then slight or indirect ventilation when temperature in the fermentor rising up naturally. The cultivation time is between 12-36 h depending on inoculum size. Inter-stage inoculum size: 1%-5%, wherein 2%-4% is most often used, That is to say, the inter-stage multiple is mostly 25%-50%. The centrifugation and rinse after the cultivation are in conventional methods.

### The Preparation of Extract of Yeast Cell Wall

Then the yeast cell is resolved by autolysis or hydrolysis, and any method of resolution disclosed in literatures can be used. In some embodiments of the invention, the yeast autolysis is undertaken under about 50° C. After resolution, the resultant cell wall is centrifuged and washed several times to remove intracellular matter and concentrate the cell wall.

The resultant yeast cell wall is transferred into a boiling vessel, boiled under 80-98° C. for 1-2 hours, then cooled to 40-60° C. and adjusted to pH 7-8. Next, a suitable proportion of biological enzyme is added, the enzyme used during this process may be selected from alkaline proteinase, mannanase, diastase and the mixture thereof, preferably alkaline proteinase. In one embodiment of the invention, 1-4%0 by weight of alkaline proteinase and 1-4%0 by weight of mannanase are used, and the enzymolysis is undertaken under 40-60° C. for 15-16 hours. Then, a hydroscopic and hydrosoluble manna oligosaccharide powder can be obtained though centrifuge separation, concentration of clear liquid and spray drying the resultant clear liquid, in sequence, The manna oligosaccharide content in the powder may reach to 40%-50% or above via the adjustment of this process.

### Provision of the Combination

The above resultant manna oligosaccharide may be mixed with clay according to above-mentioned ratio by traditional methods, for example, mechanical stirring.

The biotoxin sorbent prepared in the invention may combine with any compound feed or pet feed to form a feed product. Also the sorbent may be used as a feed additive so as to be added into any feed or daily diet. As directly mixing with feed, the biotoxin sorbent can be added at an amount of 0.25-4 kg, preferably, 0.5-3 kg, more preferably, 1-2 kg per ton of feed. The combination according to the invention can be fed to any domestic animals, aquaculture and cud chewers.

### Example

### Example 1

### Preparation

300 g of Saccharomyces cerevisiae (Dalian XingHe yeast Inc.) autolyzed under about 50° C., then the resultant cell wall was centrifuged with a centrifugal machine at a rate of 6000 rounds/min to remove intracellular matter and concentrate the cell wall. The resultant cell wall was then transferred into a boiling vessel and boiled under 80-98° C. for 1.5 hours, cooled to 40-60° C., and adjusted to pH 7-8. Next, the mixture was added 1-4‰ by weight of alkaline proteinase and 1-4‰ by weight of mannanase (based on the weight of Saccharomyces cerevisiae) and enzymolyzed at 43° C. for 15-16 hours. A hydroscopic and hydrosoluble manna oligosaccharide powder can be obtained though centrifuge separation, concentration of clear liquid and spray drying the resultant clear liquid, in sequence. The dry powder obtained after spray drying reached a content of manna oligosaccharide up to 40%-50%. Mixing the dried powder with aluminosilicate at a weight ratio of 98:2 produced a bioltoxin sorbent (Product 1).

### Example 2

### Preparation

300 g of Saccharomyces cerevisiae (Saccharomyces Cerevisiae Strain M205128 cultivated by applicant's company.) autolyzed under about 50° C., then the resultant cell wall was centrifuged with a centrifugal machine at a rate of 6000 rounds/min to remove intracellular matter and concentrate the cell wall. The resultant cell wall was then transferred into a boiling vessel and boiled under 80-98° C. for 2 hours, cooled to 40-60° C., and adjusted to pH 7-8. Next, the mixture was added 1-4‰ by weight of alkaline proteinase and 1-4‰ by weight of mannanase (based on the weight of Saccharomyces cerevisiae) and enzymolyzed at 43° C. for 15-16 hours. A hydroscopic and hydrosoluble manna oligosaccharide powder can be obtained though centrifuge separation, concentration of clear liquid and spray drying the resultant clear liquid in sequence, The dry powder obtained after spray drying reached a content of manna oligosaccharide up to 48%. Mixing the dried powder with aluminosilicate at a weight ratio of 98:2, 96:4, 92:8, 95:5, 94:6 produced correspondingly Product 2 , 3, 4, 5 and 6.

### Example 3

### Absorption Experiment In Vitro

1 mg of the above Product 1 to Product 6 were respectively added into 2 g of chicken feed containing various mycotoxin and stirred for 1 hour. Liquid chromatography was used to examine aflatoxin, zearalenone and voitoxin in the feeds, respectively, with equivalent amount of calcium aluminosilicate as a comparison, the result listed in Table 1.

TABLE 1
Absorption Effects of Bioltoxin Sorbent to Mycotoxin in
the Feed Raw Material

| | | | penicillinum | |
|---|---|---|---|---|
| | aflatoxin | ochratoxin | citrinum | zearalenone |
| | absorption | absorption | absorption | absorption |
| sorbent | ratio/% | ratio/% | ratio/% | ratio/% |
| Product 1 | 79 | 17.6 | 21.2 | 19.2 |
| Product 2 | 96.1 | 22.2 | 27.3 | 21.2 |
| Product 3 | 91.5 | 18.1 | 20.1 | 21.4 |
| Product 4 | 88 | 18.0 | 23.4 | 18.7 |
| Product 5 | 94.3 | 20.2 | 26.3 | 19.7 |
| Product 6 | 95.2 | 19.9 | 26.3 | 18.2 |
| calcium | 47 | 20.1 | 23.4 | 16.8 |
| aluminosilicate | | | | |

The experiments show that the combination according to the invention has superior absorption capability over the control, with its absorption rate positively correlative with the content of manna oligosaccharide in the product, particularly in the case of aflatoxin. Notably, the product prepared from Strain Z2.2 presents a higher absorption activity than that using commercially marketed Saccharomyces cerevisiae.

1. A biotoxin sorbent, comprising 1%-10% by weight of clay and 90%-99% by weight of yeast manna oligosaccharide, wherein the yeast manna oligosaccharide is prepared from Saccharomyces Cerevisiae Strain Z2.2.

2. The biotoxin sorbent according claim 1, comprising 4%-8% by weight of the clay and 92%-96% by weight of the yeast manna oligosaccharide.

3. (canceled)

4. A method for preparing the a biotoxin sorbent comprising the steps of:
providing yeast cell wall;
preparing manna oligosaccharide from the yeast cell wall, which includes:
i) boiling the yeast cell wall in a boiling vessel for 1 to 2 hours at 80-98° C.,
ii) enzymolyzing the yeast cell wall by using a biological enzyme selected from a group consisting of alkaline proteinase, mannanase and amylase,
iii) centrifuging to obtain enzymatic hydrolysate,
iv) concentrating the resultant clear liquid to obtain a concentrate,
v) drying the concentrate to produce a manna oligosaccharide dry powder; and
mixing the manna oligosaccharide dry powder with the clay uniformly.

5. The method according to claim 4, wherein the biological enzyme is alkaline proteinase.

6. The method according to claim 5, wherein the enzymolysis is performed at a temperature of 40° C. to 60° C. for 15-16 hours.

7. The method according to claim 4, wherein the yeast cell wall is produced by scale-up culturing a Saccharomyces cerevisiae strain using fermentable sucrose molasses and dissolving the resultant yeast cells.

8. The method according to claim 4, wherein the yeast cell wall is obtained from Saccharomyces Cerevisiae Strain Z2.2.

9-12. (canceled)

13. An animal feed, comprising a biotoxin sorbent comprised of 1%-10% by weight of clay and 90%-99% by weight of yeast manna oligosaccharide, wherein the yeast manna oligosaccharide is prepared from Saccharomyces Cerevisiae Strain Z2.2.

14. (canceled)

15. The animal feed according to claim 13, wherein the biotoxin sorbent is comprised of 4%-8% by weight of the clay and 92%-96% by weight of the yeast manna oligosaccharide.
